(19) United States
(12) Patent Application Publication
(10) Pub. No.: US 2024/0130743 A1
(43) Pub. Date: Apr. 25, 2024

(54) DEVICE FOR REMOVING DEBRIS FROM A SUBJECT

(71) Applicants: Brian Joseph Hajjar, Palisades Park, NJ (US); Michael Hajjar, Cresskill, NJ (US); Marc Hajjar, Cresskill, NJ (US)

(72) Inventor: John H. Hajjar, Cresskill, NJ (US)

(21) Appl. No.: 18/380,724

(22) Filed: Oct. 16, 2023

Related U.S. Application Data
(60) Provisional application No. 63/475,224, filed on Oct. 20, 2022.

(51) Int. Cl. A61B 17/22; A61B 90/00

(52) U.S. Cl. CPC A61B 17/22; A61B 90/361; A61B 2017/22079; A61B 2217/005; A61B 2217/007

(57) ABSTRACT

The present invention is directed to a device having at least one port and at least three lumens, wherein the lumens are for visualization, irrigation and vacuuming. The device allows for vacuuming of debris from a subject, for example renal fragments, stones and dust up to at least 1 mm in size.

The present application claims the benefit of priority under 35 U.S.C. § 119(e) of Provisional Application No. 63/475,224, filed on Oct. 17, 2022, the entire disclosure of which is hereby incorporated by reference.

### BACKGROUND

### Field of the Invention

The exemplary and non-limiting embodiments described herein relate to medical devices. The present invention is directed to a device, useful, for example, as an endoscope system with visualization, irrigation and vacuuming ability and methods for continuous evacuation of small particles, including stone dust. The device of the present invention has many uses, including use for removing debris from the renal collecting system of a subject, but may also be useful for the removal of debris from other anatomic targets, such as the bladder, gall bladder, etc.

### Description of the Related Art

Urinary stone disease (urolithiasis) is the third most common disease of the urinary tract. It is quite prevalent in the United States and in the world and its prevalence has increased over the past decades. The Southeastern part of the United States is known to be the stone belt of America. The treatment of renal stones and bladder stones are managed through medical or surgical interventions. The surgical intervention requires anesthesia and is done through the utilization of special equipment such as ureteroscopy, cystoscopy, percutaneous nephrolithotomy (PTNL) and or extracorporeal shock wave lithotripsy (ESWL). Ureteroscopy involves retrograde visualization of the renal collecting system using a rigid, semi-rigid, or flexible endoscope. The ureteroscope has a working channel that allows the introduction of a variety of instruments for stone fragmentation and removal. Percutaneous nephrolithotomy involves creating an access tract into the renal collecting system through which nephroscopy can be performed. The nephroscope has a working channel through which an intracorporeal lithotripsy device (lithotrite or laser) can be introduced. Stone fragments are removed using suction, graspers, or basket extraction. This process of removing stone fragments manually is time consuming and requires multiple passages of medical devices in and out of the patient to retrieve each fragment. This results in trauma to the ureter.

With minimally invasive percutaneous nephrolithotomy the efficiency of extraction of stone fragments and dust is lower than that of standard PTNL. Lithotripsy can be achieved with laser, or by ultrasonic or pneumatic energy. The fragmentation rate with ESWL has been around 70 to 80% and often leads to persistent residual stones. Laser tends to produce finer fragments compared to pneumatic or ultrasonic lithotripsy. The fragmentation rate with laser and ultrasound lithotripsy has been around 85 to 100%. After stone fragmentation it is generally assumed that the patient will subsequently pass the small fragments upon hydration and physical activity. The overall stone free rate has ranged from 50 to 80% range and thus multiple small fragments can remain in the kidney acting as a nidus for future stone formation. The stone free rate is defined as the detection of residual fragments <2 mm in diameter on abdominal non-contrast CT. Despite this definition of stone free, the recurrence rates are high. Stone dust where the particles <2 mm in diameter, is left to pass spontaneously.

When the fragment load is high in such procedures, multiple insertions and withdrawals of the nephroscopes to facilitate the retrieval of all the stone fragments may be required. Furthermore, some fragments as well as blood clots still require the use of the forceps. On rare occasion this may lead to slipping of guide wire.

Where suction is used to gather the stone, a separate suction device is used. The fragments are generally sucked out through the working channel of the ureteroscope Others have combined suction with the laser. Examples of such devices are the laser Suction Tube (Karl Storz®, Germany), LithAssist™ (Cook® Medical), and Suction Handpiece (HP) (Lumenis®, Israel). However, these devices are introduced through a typical rigid nephroscope working channel, have an outer diameter of more than 11 F and are not compatible with mini-nephroscopes. The large diameter of these devices may cause trauma to the ureter. Another problem often encountered by a lithotripsy endoscopist involves clogging at distal ends by stones and their fragments. Severe clogging may necessitate repeated removal, cleaning and reinsertion of the endoscope during an operation, all of which may result in unnecessary trauma and the risk of potential complications, such as infections and injury.

### SUMMARY OF THE INVENTION

The present invention relates to a technically feasible and efficacious flexible device with irrigation and vacuum suction systems to remove debris, such as stone dust and stone fragments from a subject, for example, from the renal collecting system of a subject.

An object of the present invention is to provide a device which will be utilized after fragmenting the stones, such as by laser lithotripsy or ESWL to vacuum the stone dust and stone fragments.

In an embodiment, the device will be inserted into the subject, for example, into the ureter of a subject, similar to insertion of a flexible ureteroscope, and once it is positioned, it can then vacuum the stone dust and stone fragments. In one embodiment, the device can vacuum fragments up to about 1 mm in size. The device comprises a handle which remains outside the body. The handle includes at least one port. In a preferred embodiment, the handle includes a first, second and third port, and an elongated shaft extending form the handle to a distal end having in an embodiment, three lumens corresponding to each port. In an embodiment, one of the ports is a fluid port, which may be connected to a fluid source to irrigate the target surgical site through the elongate shaft. In another embodiment, another port is a vacuum port which may be connected to a vacuum source and which is capable of applying suction through the elongated shaft. In a further embodiment, the device further includes at least one optics port which may be attached to a visualization apparatus. The device further includes a tip at the distal end, opposite to the handle. In an embodiment, the tip comprises three lumens, which for example, correspond to each of the three ports.

In a preferred embodiment, the device of the present invention has three portals for visualization, irrigation, and vacuuming. These portals include (1) a vacuum port, (2) an irrigation port, and (3) an optical port, each of which may include 1) at least one vacuum lumen 2) at least one irrigating lumen 3) and at least one optics lumen for visualization.

In an embodiment of the invention, the device is small enough to enter an orifice of the subject. For example, the device is small enough to enter the ureter and the kidney (calyx) similar to the flexible ureteroscopes currently available. The device may also enter the subject via a small incision.

In an embodiment of the invention, the device includes a handle portion (I), which is intended to remain outside of the body of a subject, and shaft portion (H) which may be partially or fully inserted into the body of a subject. The shaft portion further includes a tip portion (J).

In a particularly preferred embodiment, the length of the device shaft portion is between 40-45 cm and the outer diameter of the shaft portion is about 1° F. or less, preferably about 9.5 F or less and the out diameter of the vacuum port is 5 or less, preferably, 4.5 or less. In one embodiment, the out diameter of the tip is 7.7 F, and the diameter of the shaft is 9.5 F (see, e.g. FIG. 6).

As used herein the unit “F” or “Fr” refers to the catheter gauge system French size, which is based upon a measurement of the external diameter of a catheter tube. The French size of a catheter is determined by a simple multiplication of 3 (diameters in millimeters multiplied by 3=the French size). For example, if a catheter has a diameter of 4.7 millimeters, then the French (Fr) size is 14.

In an embodiment of the invention, the device has the ability to evacuate debris, such as, e.g., stone dust and stone fragments, from a subject. In a preferred embodiment, the device removes debris up to 1 mm in size. Such sand like-granules, when not removed, form the nidus for new stones. An advantage of the device of the present invention is the elimination of the need for the patient to pass small stone fragments as these are removed by the vacuum source. Accordingly, there is preferably minimal to no retained or remaining stone. This greatly diminishes the reoccurrence of stones as the nadir for new stone formation is removed.

The device is also capable of ensuring decreased retropulsion or unintentional migration of the stones to other parts of the pelvicalyceal system such as the ureter and bladder.

In some embodiments, the device may be used to suction debris other than stone dust and stone fragments, including renal stone dust and stone fragments. Such debris may include, for example, tissue, cells, bone, blood, clots, tumors, polyps, lesions, strictures, cysts, gallbladder or gallbladder stones, sludge or sand, or foreign bodies, such as debris caused by microbial infections.

The device is effective in clearing debris, such as stone fragments and stone dust, with less complications such as decreased hemoglobin, less bleeding and/or transfusion rates, lower infection rates (e.g., urinary tract infection rates), and a lower likelihood of post-op fever. In addition, pain scores as assessed by the visual analogue score or other tools may be lower and hospitalizations shorter. This is because of the smaller lumen outer diameter and shaft of the device of the present invention.

In a preferred embodiment, the vacuum portal of the invention is 4 F, more preferably, 3.5 F. The vacuum lumen is preferably IF.

The vacuum portal will be the largest portal on the scope. However, the device will be compatible with mini-nephroscopes making it useful for pediatric patients.

In one embodiment, the vacuum portal or vacuum lumen of the device is connected it to an outside vacuum source. In one embodiment, the vacuum source provides a suction pressure capacity of 200 mmHg (1 mmHg=0.133 kPa). The vacuum source connected to the port applies suction through an elongated shaft and a shaft lumen of the device. In one embodiment, the device provides for the vacuum source to be clutch controlled so that a variable suction of 0-200 mmHg can be achieved, and the device handle, for example, provides a locking mechanism in case the surgeon wants to have constant full power of suction. The clutch control enables the surgeon to deliver a requisite amount of suction, and it is not an all or none phenomenon.

In some embodiments the device includes a pressure transducer. The pressure transducer is configured to be capable of measuring pressure within the surgical site.

In some embodiments the negative pressure is set from −25 k Pa to −4 k Pa in continuous mode for suction of debris.

In some embodiments the vacuum may be a wall vacuum or wall suction. In other embodiments, the vacuum will be a hand-held vacuum or bulk vacuum.

In some embodiments, the irrigation port is configured to provide irrigation by, e.g., gravity or pressurized irrigation. In an embodiment, the irrigation port provides irrigation fluid. The irrigation fluid may be any fluid suitable for irrigation in a subject, e.g., normal saline or other isotonic fluid.

In a preferred embodiment, the vacuum should not be too strong so that it will harm the lining of the collecting system of the subject, such as the collecting system of the kidneys, bladder or gall bladder.

The vacuum power preferably is strong enough to evacuate the debris, such as those at least up to 1 mm in size, without injuring the subject, for example, without injuring urothelium, causing ureteral stricture, lacerating or traumatizing the ureter, causing ureteral pseudochannels, avulsions or perforations, causing perineal hematomas or damaging the surrounding tissues in the kidney.

In another embodiment, the device of the invention is flexible up to 45 degrees. Preferably, the device will not lose its suction capacity once it is flexed to that degree. In other embodiments the device may be semi-rigid or even rigid.

According to the present invention as described above, the device can be disposable or partially disposable.

Another advantage of the present invention may be a decrease in operation time. The device of the invention enhances the speed for which the procedure is completed. The entire procedure can take between approximately 10-30 minutes whereas the procedure may take hours using methods known to those skilled in the art.

It is an object of the embodiments of the present invention to have radiographic and or ultrasound complete clearance (stone-free rate) in a larger number of patients. Preferably, at one month post procedure, patients have more complete clearance. The device of the present invention preferably can decrease the need for other procedures, such as the need for nephrostomy placement.

At follow-up, stones larger than 18 mm may be less in patients where the device was used. This is because of the vacuuming of fragments and dust less than 1 mm.

In some embodiments, the device may be used in stone procedures such as mini-percutaneous nephrolithotomy, ureteroscopy, cystoscopy, percutaneous nephrolithotomy (PTNL) and or extracorporeal shock wave lithotripsy (ESWL).

### BRIEF DESCRIPTION OF THE DRAWINGS

FIG. 1 illustrates various particle sizes for renal stone fragments and dust.

FIG. 2 shows a plan view of an exemplary embodiment of a device according to the present disclosure.

FIG. 3 shows a side view of a further exemplary embodiment of a device according to the present disclosure.

FIG. 4 depicts a front view of an exemplary tip of a device according to the present disclosure.

FIG. 5 depicts a front view of a further exemplary tip of a device according to the present disclosure.

FIG. 6 depicts a plan view of an exemplary tip of the device according to the present invention.

FIG. 7 depicts a cross-sectional view of an exemplary shaft of the device according to the present disclosure.

### DETAILED DESCRIPTION OF THE INVENTION

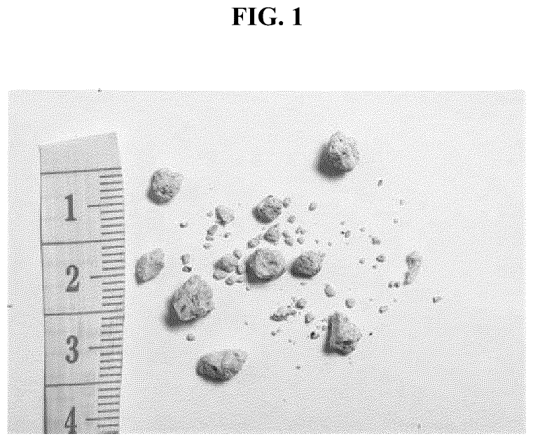

Both dusting and fragmentation with extraction approaches to ureteroscope stone treatment are effective. In fact, there is little evidence that one approach is better than the other. The goal of dusting is to fragment the stone into tiny pieces that resemble “dust” and thus can pass spontaneously. Although dusting tends to be associated with shorter procedure times and a lower risk of ureteral damage, this approach may place the patient at increased risk for future stone events should all of the resultant debris not be expelled from the collecting system. FIG. 1 illustrates various particles sizes for fragments and dust for renal stones.

The disclosure provides devices and methods for the removal of stone fragments and debris from a patient's body, especially the kidney, ureter and bladder. The devices and methods are also useful for the removal of stone fragments and debris from other parts of a patient's body, e.g., the gall bladder.

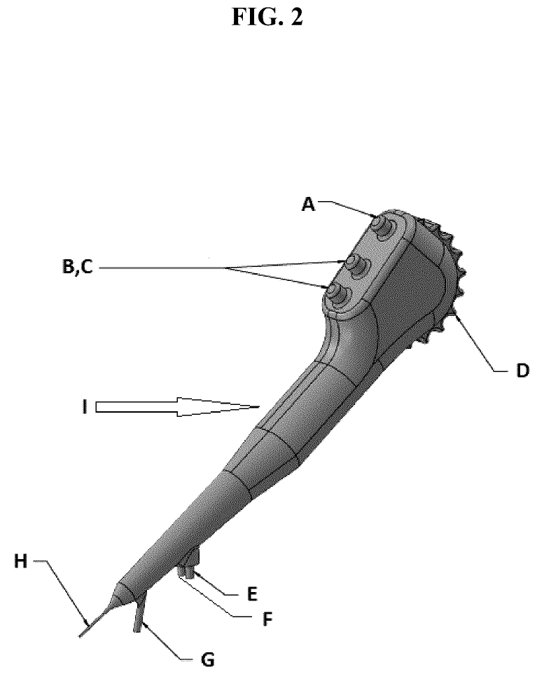

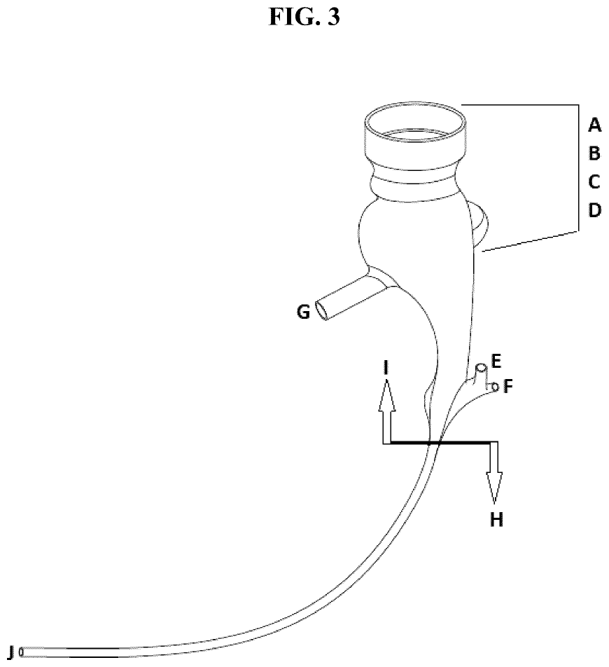

In one embodiment, proximal to the device tip are three lumens. Each lumen may be configured to be partially inserted into the patient's body, such as through the patient's urethra, into the bladder, and possibly into the ureter where the stone fragments and dust can be visualized. The lumens originate from the at least one port. In a preferred embodiment, the lumens originate from a vacuum port (E), fluid port (F) and optics port (G) (see, e.g., FIGS. 2 and 3).

In some embodiments, the lumens have indica of identification to differentiate them from each other. For example, they may be color-coded, prominently labeled or bear discernible markings.

FIG. 2 depicts an exemplary embodiment of the device of the present invention having a handle I and a shaft H and wherein A is attached to a camera, B and C are controls for irrigation (from the fluid port) and suction (from the vacuum port), E is a vacuum port, F is a fluid port, G is an optics port and D is a deflection wheel for controlling the insertion of the shaft/lumens into a subject.

The vacuum lumen can be controlled with an actuator, on/off button, foot pedal or other similar mechanism. Debris is expirated through the vacuum lumen. There is no need for a basket device to be inserted through the vacuum lumen to collect the debris as the fragments and debris go directly into the vacuum. The vacuum lumen is connected to a vacuum source via a supply line (e.g., tubing).

The device is connected to suction with a suction pressure capacity of 200 mmHg (1 mmHg=0.133 kPa) so that a suction of 0-200 mmHg can be achieved. In some embodiments, the suction pressure is preferably between 20-150 mmHg, or more preferably between 40-110 mmHg. In the most preferred embodiment, the suction pressure is 95 mmHg. The suction must be sufficient to vacuum up the debris, stones, fragments, and dust but not so strong as to damage the tissues of the subject, such as the tissues of the kidney.

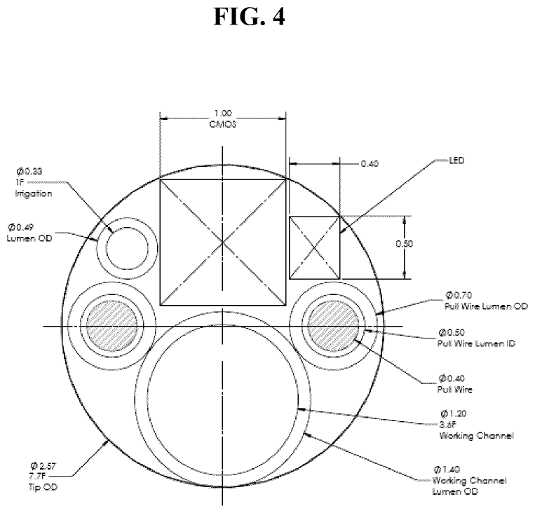

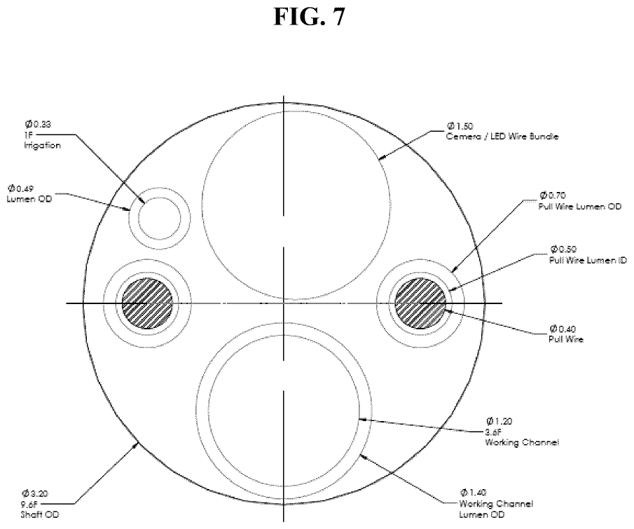

In a preferred embodiment, the length of the shaft of the device is between 35-45 cm, the outer diameter of the shaft is 10 F or less with an inner working channel of or vacuum lumen of 5 F or less, preferably the shaft is 9.5 F and the vacuum lumen is 4.5 F. The length of the handle of the device, which includes the working elements, is preferably about 25 cm. In a preferred embodiment, the length of the shaft of the device is between 38-43 cm, the outer diameter of the shaft is between 6 and 8 F, with a working channel of 3.6 F (see, e.g., FIGS. 4 and 7). In a more preferred embodiment, the length of the inner portion of the device is between 41-44 cm, the outer diameter of the device (tip) is 8 F with a working channel of 3 F. In a most preferred embodiment, the length of the shaft is 40 cm, the outer diameter of the device is 8 F and the length of the handle, including the working elements, is 25 cm.

In a preferred embodiment, the diameter of the vacuum port is 3.5 F, the water or irrigation port is 1.9 F and the optical port is 2.6 F.

In a preferred embodiment, the lumens are made of flexible materials. Any material (e.g. plastic) that provides flexibility to 45 degrees may be used.

The vacuum lumen conduit can be made of a variety of flexible materials, such as plastics or elastic alloy such as Nitinol. A preferred embodiment is polypropylene lumen. To improve the lumen's resistance against kink-formation or against collapse under vacuum pressure, and to preserve flexibility in the meantime, the lumen can be braided or wound with fibers made of materials such as metals or plastics. The lumen may have coatings on its inside or outside for various purposes, for example, for protection against corrosion by body fluids such as urine or blood, for insulation against high energy emitted from a laser, or with anti-microbials to hinder infection.

The present invention overcomes disadvantages associated with current strategies and provides tools, and other methods to facilitate and simplify removal of stone fragments and dust up to, preferably, at least 1 mm in size.

The surgeon may adjust the rotary knob to control the negative pressure and actively control the pressure of the suction of stones for simultaneous reduction of the pressure inside the subject, for example, in the pelvis, to achieve active suction of the stones.

Certain embodiments of the present invention include, but are not limited to, the method of the invention as follows:
1) Patient will be placed in a supine lithotomy position. Patient will undergo intravenous sedation.
2) The perineum will be prepped and traced in a sterile manner.
3) The urethra will be intubated directly with the flexible (or ridged) device which will be introduced into the ureteral orifice.
4) The device will be inserted into the ureter (either under direct vision or under fluoroscopic guidance).
5) The ureteral orifices will be intubated with a flexible glidewire (aka pull wire) as well. By doing so, the ureter will then be directly intubated with the device under direct vision or with the guidance of a fluoroscope. This will be done over the glidewire to reduce injury or any inadvertent placement outside the ureteral body.
6) Once the device is in the pelvic region, the glide wire is then removed and the entire kidney (including the upper, lower and middle) pole of the calyceal system will be directly visualized and any stone fragments or encountrants less than 1 mm in size will be vacuumed and removed.

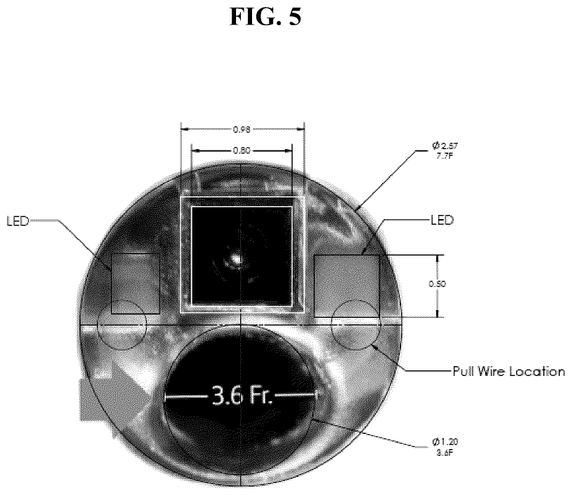

The glidewire (also referred to herein as a pull wire) is used to minimize trauma and to ensure the device is within the ureter. An exemplary embodiment of a pull wire location within the device of the present disclosure is depicted, e.g., in FIGS. 4, 5 and 7

The preferred isotonic irrigation solution is normal saline.

As used herein, the terms endoscope, nephroscope and uteroscope are used interchangeably without limitation. Other improved scopes may be employed such as hysteroscopes and cystoscopes, and the like, and any other scope that can be inserted into the body with a lumen.

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example.

While in a preferred embodiment, the device is for use in the urinary system, the device may be used in any anatomic target where there is a need to vacuum and remove debris.

According to one embodiment the device may be used to remove debris with consistencies ranging from soft to hard (including bone). This debris capable of being removed may be renal or urinary dust or stones, tissue, cells, bone, blood, clots, tumors, polyps, lesions, strictures, cysts, gallbladder or gallbladder stones, sludge or sand, or foreign bodies, such as debris caused by microbial infections. FIG. 1 depicts renal stones and fragments. Additionally, the device can be used for orthopedic applications and endoscopic applications such as arthroscopy and endoscopic retrograde cholangiopancreatography (ERCP). The device may also be used in various other procedures such as, for example, hysteroscopies, cystoscopies, etc. The device may be used with other devices such as cystoscopes, hysteroscopes or any other device with an elongated shaft with a lumen inserted into the body.

According to one aspect of the invention, the device is disposable, or partially disposable. The lumens may be single use. A used lumen is disconnected from the handle and disposed and a new lumen is connected to the handle for the next procedure.

The energy source for the device may be electrical, battery, remote or manual.

In another embodiment, the device has a fourth lumen directed to a different functionality such as a laser to apply pulsed laser energy to the stone to fragment the stone into smaller pieces. The fourth lumen is referred to herein as a fragmentation lumen and may be included in a further port, the fragmentation port.

The device may be configured to emit a pulse of laser energy. Lasers that can be used with the device include, lasers that are effective in fragmentation of stones of varying hardness and have a low penetration depth, but are not limited to, Holmium laser, holmium:yttriumaluminum-garnet (HoYAG) laser, Lumenis 120 W laser, thulium fiber laser, pulsed dye laser, alexandrite laser, neodymium laser. Any laser that is suitable for the treatment of ablation of stones may be used.

The fluid port (F) is connected to a fluid source which pumps fluid to irrigate and dislodge debris from the anatomical site. Irrigation devices may include aspiration devices or the like. Fluid flow may be controlled with an on/off button or a foot pedal. The irrigating lumen may be filled with irrigation fluid, water or other liquids or materials to fill the cavity. The vacuum and irrigation can be turned on as needed and off if not needed.

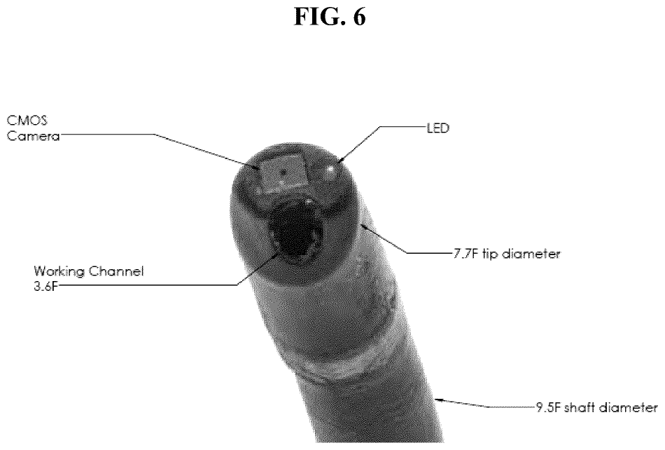

One or more ancillary devices such as cameras, digital imaging units, lights, illumination fibers, reflectors, mirrors, prisms, magnifiers, lenses, other imaging devices may be coupled to the device. The image of the stone fragments and dust may be displayed on a screen. In an embodiment, the device includes an image capture control mechanism (A in FIG. 2). The device also includes a camera and light (LED) source as shown in FIGS. 4-7.

In one aspect, the devices of the invention can also be equipped with structures such as barriers or shields in the distal region of the suction conduit to help block large particles which have the capability of clogging the device.

The device can be controlled manually, or via a controller, sensors, pressure transducers, or connected to a computer processor having a pointing device, memory, and capable of performing various functions such as calculations or computations.

In a preferred embodiment, the subject is a human adult patient. In some embodiments, the subject is a human pediatric patient. In some embodiments, the subject may be an animal.

The present disclosure will be described with respect to particular embodiments and with reference to certain drawings, but the present disclosure is not limited thereto but only by the claims. The drawings described are only schematic and are non-limiting.

Reference throughout this specification to “one embodiment” or “an embodiment” means that a particular feature, structure or characteristic described in connection with the embodiment is included in at least one embodiment of the present disclosure. Thus, appearances of the phrases “in some embodiments,” “in one embodiment,” or “in an embodiment” in various places throughout this specification are not necessarily all referring to the same embodiment, but may. Furthermore, the particular features, structures, or characteristics may be combined in any suitable manner, as would be apparent to one of ordinary skill in the art from this disclosure, in one or more embodiments.

The disclosure presented herein is believed to encompass at least one distinct invention with independent utility. While the at least one invention had been disclosed in exemplary forms, the specific embodiments thereof as described and illustrated herein are not to be considered in a limiting sense, as numerous variation are possible. Equivalent changes, compositions and methods may be made within the scope of the present disclosure, achieving substantially similar results. The subject matter of the at least one invention includes all novel and on-obvious combinations and sub-combinations of the various elements, features, functions and/or properties disclosed herein and their equivalents.

What is claimed:

1. A device for the removal of debris from a subject, wherein the device comprises at least one port and wherein the at least one port comprises at least one fluid lumen, at least one vacuum lumen and at least one optics lumen.

2. The device of claim 1, wherein the at least one port comprises a fluid port, a vacuum port and an optics port, each of which independently and respectively houses the fluid lumen, vacuum lumen and optics lumen.

3. The device of claim 1, wherein the debris comprises renal or urinary dust or stones, tissue, cells, bone, blood, clots, tumors, polyps, lesions, strictures, cysts, gallbladder or gallbladder stones, sludge or sand, or foreign bodies, such as debris caused by microbial infections, or combinations thereof.

4. The device of claim 2, wherein the device further comprises a shaft and a handle.

5. The device of claim 4, wherein the shaft includes a tip and the handle includes the ports and at least one controller.

6. The device of claim 1, wherein the device is connected to a vacuum source.

7. The device of claim 6, wherein the vacuum source is connected to the vacuum lumen.

8. The device of claim 1, wherein the fluid port is capable of delivering fluid to the subject at the location of the debris.

9. The device of claim 1, wherein the optics lumen of the device is connected to a visualization apparatus.

10. The device of claim 9, wherein the visualization apparatus comprises at least one of a light source, a camera, and a display.

11. The device of claim 1, wherein the lumens are flexible.

12. The device of claim 1, wherein the device is disposable.

13. The device of claim 4, wherein the shaft comprises a diameter of at least 7 F.

14. The device of claim 4, wherein the shaft has a length of about 40 cm.

15. The device of claim 4, wherein the handle has a length of about 25 cm.

16. The device of claim 1, wherein the lumens are comprised of a flexible material.

17. The device of claim 1, further comprising a fragmentation lumen.

18. The device of claim 17, wherein the fragmentation lumen is connected to a fragmentation device.

19. The device of claim 18, wherein the fragmentation device is a laser, an ultrasonic energy device or a pneumatic energy device.

20. The device of claim 1, wherein the device further comprises a pull wire.

21. A method of treating a subject having debris in at least one cavity of the subject's body, the method comprising:
a. Inserting a device into the subject, wherein the device comprises at least one port and a fluid lumen, a vacuum lumen and an optics lumen,
b. Visualizing the debris with the optics lumen,
c. Providing fluid with the fluid lumen to the cavity in the subject's body where the debris is located,
d. removing the fluid in the cavity with the vacuum lumen, wherein the removal of the fluid results in the removal of the debris from the subject.
